SEQUENCE LISTING (17 sequences)

SEQ ID NO: 1 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 2 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 3 - protein, 832 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 4 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 5 - protein, 701 aa, Artificial Sequence
  Description of Artificial Sequence Synthetic polypeptide
MAIRRCWPRVVPGPALGWLLLLLNVLAPGRASPRLLDFPAPVCAQEGLSCRVKNSTCLDDSWIHPKNLTPSSPKNIYINLSVSSTQHGELVPVLHVEWTLQTDASILYLEGAELSVLQLNTNERLCVKFQFLSMLQHHRKRWRFSFSHFVVDPGQEYEVTVHHLPKPIPDGDPNHKSKIIFVPDCEDSKMKMTTSCVSSGSLWDPNITVETLDTQHLRVDFTLWNESTPYQVLLESFSDSENHSCFDVVKQIFAPRQEEFHQRANVTFTLSKFHWCCHHHVQVQPFFSSCLNDCLRHAVTVPCPVISNTTVPKPVADYIPLWVYGLITLIAILLVGSVIVLIICMTWRLSGADQEKHGLGSENLYFQYRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 6 - protein, 633 aa, Artificial Sequence
  Description of Artificial Sequence Synthetic polypeptide
MGPQAAAGRMILLVVLMLSAKVGSGALTSTEDPEPPSVPVPTNVLIKSYNLNPVVCWEYQNMSQTPIFTVQVKVYSGSWTDSCTNISDHCCNIYEQIMYPDVSAWARVKAKVGQKESDYARSKEFLMCLKGKVGPPGLEIRRKKEEQLSVLVFHPEVVVNGESQGTMFGDGSTCYTFDYTVYVEHNRSGEILHTKHTVEKEECNETLCELNISVSTLDSRYCISVDGISSFWQVRTEKSKDVCIPPFHDDRKDSIWILVVAPLTVFTVVILVFAYWYTKKNSFKRKSIMLLGSENLYFQYRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 7 - protein, 620 aa, Artificial Sequence
  Description of Artificial Sequence Synthetic polypeptide
MLSRLLPFLVTISSLSLEFIAYGTELPSPSYVWFEARFFQHILHWKPIPNQSESTYYEVALKQYGNSTWNDIHICRKAQALSCDLTTFTLDLYHRSYGYRARVRAVDNSQYSNWTTTETRFTVDEVILTVDSVTLKAMDGIIYGTIHPPRPTITPAGDEYEQVFKDLRVYKISIRKFSELKNATKRVKQETFTLTVPIGVRKFCVKVLPRLESRINKAEWSEEQCLLITTEQYFTVTNLSILVISMLLFCGILVCLVLQWYIRHPGKLPTVLVFKKPLGSENLYFQYRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 8 - protein, 592 aa, Artificial Sequence
  Description of Artificial Sequence Synthetic polypeptide
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGLGSENLYFQLRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 9 - protein, 750 aa, Artificial Sequence
  Description of Artificial Sequence Synthetic polypeptide
MPVSWFLLSLALGRNPVVVSLERLMEPQDTARCSLGLSCHLWDGDVLCLPGSLQSAPGPVLVPTRLQTELVLRCPQKTDCALCVRVVVHLAVHGHWAEPEEAGKSDSELQESRNASLQAQVVLSFQAYPIARCALLEVQVPADLVQPGQSVGSAVFDCFEASLGAEVQIWSYTKPRYQKELNLTQQLPDCRGLEVRDSIQSCWVLPWLNVSTDGDNVLLTLDVSEEQDFSFLLYLRPVPDALKSLWYKNLTGPQNITLNHTDLVPCLCIQVWSLEPDSERVEFCPFREDPGAHRNLWHIARLRVLSPGVWQLDAPCCLPGKVTLCWQAPDQSPCQPLVPPVPQKNATVNEPQDFQLVAGHPNLCVQVSTWEKVQLQACLWADSLGPFKDDMLLVEMKTGLNNTSVCALEPSGCTPLPSMASTRAARLGEELLQDFRSHQCMQLWNDDNMGSLWACPMDKYIHRRWVLVWLACLLLAAALFFFLLLKKDRRKAARGSRTALGSSLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTSQEAQQWVSGWRLNADSVLWGGHKVFMSKPEEPFQPVKEATQLMNELVYSYPYDVPDYA

SEQ ID NO: 10 - protein, 572 aa, Artificial Sequence
  Description of Artificial Sequence Synthetic polypeptide
MLLVLLILAASCRSALPREPTIQCGSETGPSPEWMVQHTLTPGDLRDLQVELVKTSVAAEEFSILMNISWILRADASIRLLKATKICVSGKNNMNSYSCVRCNYTEAFQSQTRPSGGKWTFSYVGFPVELSTLYLISAHNIPNANMNEDSPSLSVNFTSPGCLNHVMKYKKQCTEAGSLWDPDITACKKNEKMVEVNFTTNPLGNRYTILIQRDTTLGFSRVLENKLMRTSVAIPVTEESEGAVVQLTPYLHTCGNDCIRREGTVVLCSETSAPIPPDDNRRMLGGWLPLFLVLLVAVWVLAAGIYLTWRQGRSTKTSFPISGSSLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTSQEAQQWVSGWRLNADSVLWGGHKVFMSKPEEPFQPVKEATQLMNELVYSYPYDVPDYA

SEQ ID NO: 11 - protein, 532 aa, Artificial Sequence
  Description of Artificial Sequence Synthetic polypeptide
MRPLPLWLPSLLLCGLGAAASSPDSFSQLAAPLNPRLHLYNDEQILTWEPSPSSNDPRPVVYQVEYSFIDGSWHRLLEPNCTDITETKCDLTGGGRLKLFPHPFTVFLRVRAKRGNLTSKWVGLEPFQHYENVTVGPPKNISVTPGKGSLVIHFSPPFDVFHGATFQYLVHYWEKSETQQEQVEGPFKSNSIVLGNLKPYRVYCLQTEAQLILKNKKIRPHGLLSNVSCHETTANASARLQQVILIPLGIFALLLGLTGACFTLFLKYQSRVKYWFQAPPNIGSSLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTSQEAQQWVSGWRLNADSVLWGGHKVFMSKPEEPFQPVKEATQLMNELVYSYPYDVPDYA

SEQ ID NO: 12 - protein, 508 aa, Artificial Sequence
  Description of Artificial Sequence Synthetic polypeptide
MSWAPSVAGWLGGFLLVPALGMIPPPEKVRMNSVNFKNILQWEVPAFPKTNLTFTAQYESYRSFQDHCKRTASTQCDFSHLSKYGDYTVRVRAELADEHSEWVNVTFCPVEDTIIGPPEMQIESLAESLHLRFSAPQIENEPETWTLKNIYDSWAYRVQYWKNGTNEKFQVVSPYDSEVLRNLEPWTTYCIQVQGFLLDQNRTGEWSEPICERTGNDEITPSWIVAIILIVSVLVVFLFLLGCFVVLWLIYKKTKHTFGSSLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTSQEAQQWVSGWRLNADSVLWGGHKVFMSKPEEPFQPVKEATQLMNELVYSYPYDVPDYA

SEQ ID NO: 13 - protein, 499 aa, Artificial Sequence
  Description of Artificial Sequence Synthetic polypeptide
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGGSSLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTSQEAQQWVSGWRLNADSVLWGGHKVFMSKPEEPFQPVKEATQLMNELVYSYPYDVPDYA

SEQ ID NO: 14 - protein, 371 aa, Artificial Sequence
  Description of Artificial Sequence Synthetic polypeptide
MPEPAKSAPAPKKGSKKAVTKAQKKGGKKRKRSRKESYSIYVYKVLKQVHPDTGISSKAMGIMNSFVNDIFERIAGEASRLAHYNKRSTITSREIQTAVRLLLPGELAKHAVSEGTKAITKYTSAKDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 15 - protein, 239 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 16 - protein, 60 aa, Artificial Sequence
  Description of Artificial Sequence Synthetic polypeptide
  SITE (1)..(5): This region may encompass 2-5 residues
  SITE (1)..(60): This sequence may encompass 1-10 “(Gly)n Ser” repeating units where n=2-5
  SITE (7)..(11): This region may encompass 2-5 residues
  SITE (13)..(17): This region may encompass 2-5 residues
  SITE (19)..(23): This region may encompass 2-5 residues
  SITE (25)..(29): This region may encompass 2-5 residues
  SITE (31)..(35): This region may encompass 2-5 residues
  SITE (37)..(41): This region may encompass 2-5 residues
  SITE (43)..(47): This region may encompass 2-5 residues
  SITE (49)..(53): This region may encompass 2-5 residues
  SITE (55)..(59): This region may encompass 2-5 residues
GGGGGSGGGGGSGGGGGSGGGGGSGGGGGSGGGGGSGGGGGSGGGGGSGGGGGSGGGGGS

SEQ ID NO: 17 - protein, 6 aa, Artificial Sequence
  Description of Artificial Sequence Synthetic 6xHis tag
HHHHHH

(19) United States
(12) Patent Application Publication
(10) Pub. No.: US 2024/0053325 A1
(43) Pub. Date: Feb. 15, 2024

(54) NOVEL SCREENING PLATFORM TO IDENTIFY IMMUNE MODULATORY AGENTS

(71) Applicant: PRESIDENT AND FELLOWS OF HARVARD COLLEGE, Boston, MA (US)

(72) Inventors: Jun HUH, Cambridge, MA (US); Guangqing LU, Cambridge, MA (US)

(73) Assignee: PRESIDENT AND FELLOWS OF HARVARD COLLEGE, Boston, MA (US)

(21) Appl. No.: 18/266,423

(22) PCT Filed: Dec. 10, 2021
(86) PCT No.: PCT/US2021/062761

§ 371 Date: Jun. 9, 2023

Related U.S. Application Data
(60) Provisional application No. 63/124,251, filed on Dec. 11, 2020.

(51) Int. Cl. G01N 33/50; C12N 9/50; C12Q 1/6897

(52) U.S. Cl. CPC G01N 33/5023; C12N 9/506; C12Y 304/22044; C12Q 1/6897; G01N 2333/7155

(57) ABSTRACT

Provided herein is a reporter system for identifying a cytokine receptor modulator and uses thereof.

### CROSS-REFERENCE TO RELATED APPLICATIONS

This application is a 35 U.S.C. § 371 National Phase Entry Application of International Patent Application No. PCT/US2021/062761 filed on Dec. 10, 2021, which designated the U.S., and which claims benefit under 35 U.S.C. § 119(e) of U.S. Provisional Application No. 63/124,251, filed Dec. 11, 2020, content of each of which is incorporated herein by reference in its entireties.

### SEQUENCE LISTING

The instant application contains a Sequence Listing which has been submitted electronically in ASCII format and is hereby incorporated by reference in its entirety. Said ASCII copy, created on Feb. 3, 2022, is named 002806-099140WOPT_SL.txt and is 72,434 bytes in size.

### TECHNICAL FIELD

The technology described herein relates to screening for and identifying immune modulatory agents and uses thereof.

### BACKGROUND

Immune cells often exert their effects by secreting cytokines. Cytokines then bind to their cognate receptors expressed on the membranes of target cells, binding of which initiates a downstream signaling cascade leading to various biological effects. Under pathological conditions, Th17 cells and IL-17a, and other cytokines can promote inflammatory and autoimmune diseases. Modulating cytokine activity with small molecules may have therapeutic value not only in controlling autoimmunity but also in preventing and/or therapeutically treating certain types of neurodevelopmental disorders. There is currently an unmet need for platforms and screening tools that identify immunomodulatory therapeutics for autoimmune and neurodevelopmental disorders.

### SUMMARY

The compositions and methods described herein are based in part on the discovery of a screening platform that can be used to identify immune modulatory agents including, but not limited to, small molecules and proteins that modulate cytokine signaling.

In one aspect, provided herein is a reporter system for identifying a cytokine receptor modulator. Generally, the system comprises two or more fusion polypeptides. A first polypeptide of the reporter system comprises a domain comprising a first subunit of a cytokine receptor and a second domain comprising at least a transcription activation domain and a DNA-binding domain of a transcription factor. The first and the second domain are linked by a linker comprising a cleavable group. For example, the linker comprises a protease cleavage site.

A second fusion polypeptide of the reporter system comprises a first domain comprising a second subunit of the cytokine receptor and a second domain comprising at least a catalytic domain of a protease. The first and second domains can be linked by a linker. The linker can be a flexible linker.

In some embodiments of any one of the aspects, the reporter system also comprises a reporter gene for detecting an activity of the cytokine receptor. Generally, the reporter gene is linked to an inducible promoter.

In another aspect, provided herein is a cell comprising the reporter system provided herein.

In another aspect, provided herein is a polynucleotide encoding one, two or three of the first fusion polypeptide, the second fusion polypeptide and the reporter gene provided herein.

The polynucleotide can be comprised in a vector and/or in a cell. Thus, in another aspect, provided herein is a cell comprising the polynucleotide provided herein.

The reporter system described herein can be used for identifying agents that can modulate immune response. According, in yet another aspect, provided herein is a method for identifying a test agent that modulates an immune response. Generally, the method comprises contacting a test agent with a cell comprising a reporter system provided herein and detecting or measuring an expression level of the reporter gene. The expression level of the reporter can be compared to a control or reference level and an increase in the expression level of the reporter gene relative to the control or reference level indicates the agent modulates an immune response.

### A BRIEF DESCRIPTION OF THE DRAWINGS

This application file contains at least one drawing executed in color. Copies of this patent application publication with color drawings will be provided by the Office upon request and payment of the necessary fee.

FIG. 1 is a schematic of an IL-17RA/RC reporter design according to an embodiment of the disclosure. IL-17a binding to the heterodimeric complex of IL-17RA and IL-17RC leads to the recruitment of two protein subunits, resulting in the release of tTA from the C-terminus of modified IL-17RA. Cleaved tTA then enters the nucleus and stimulates GFP reporter gene activity.

FIG. 2 shows the specificity of an exemplary IL-17RA/RC reporter assay of the disclosure. With 24 h stimulation, IL-17A, but not IL-17B/IL-17C/IL-17D/IL-17E, significantly increased the percentage of GFP⁺ cells (from 1% to 60%). Tumor necrosis factor alpha (TNFa) served as a negative control and failed to increase GFP expression. Doxycycline was used as a negative regulator of the GFP reporter. IL-17A-F: 100 ng/ml, TNFa: 5 ng/ml, doxycycline: 1 μg/ml.

FIG. 3 shows dose-dependent activation of an exemplary IL-17RA/RC reporter assay of the disclosure. The IL-17RA/RC reporter cells were incubated with different amounts of IL-17A, IL-17C and IL-17F for 24 hours. Both IL-17A and IL-17F, but not IL-17C, activate the GFP reporter system in a dose-dependent manner.

FIG. 4 shows the specificity of an exemplary IL-17RA/RB reporter assay of the disclosure. With 24 h stimulation, IL-17E, but not IL-17A/IL-17B/IL-17C/IL-17D/IL-17F/IL-17A/F, significantly increased the percentage of GFP⁺ cells (from 1% to 80%). Tumor necrosis factor alpha (TNFa) served as a negative control and failed to increase GFP expression. Doxycycline was used as a negative regulator of the GFP reporter. IL-17A-F: 100 ng/ml, TNFa: 5 ng/ml, doxycycline: 1 μg/ml.

FIGS. 5A-5F show dose dependent activation of exemplary IL-10/IFN-γ/TNF-α reporters of the disclosure. IL-10/IFN-γ/TNF-α reporters were incubated for 24 hours with different amounts of IL-10 (FIGS. 5A and 5B), IFN-γ (FIGS. 5C and 5D) or TNF-α (FIGS. 5E and 5F).

FIGS. 6A-6F show small molecule screen using some exemplary reporter assays of the disclosure. FIGS. 6A-6C, Agonist screens (23,031 compounds) with the IL-17A and IL-10 reporters. FIGS. 6D-6F, Antagonist screens with the IL-17A and TNF-α reporters. Two replicate screens display consistent and reproducible results (FIGS. 6A, 6B, 6D and 6E). N, negative controls with no added small molecules; P, positive controls with the respective cytokines (FIGS. 6A-6C) or cytokine blocking antibodies (FIGS. 6D-6F); X, individual compounds; Blue circles, identified hits that will be tested in cherry-pick screens.

### DETAILED DESCRIPTION

The compositions and methods described herein are based in part on the discovery of a screening platform that can be used to identify immune modulatory agents including, but not limited to, small molecules and proteins that modulate cytokine signaling.

Provided herein are methods, systems, and compositions for use in detecting an agent the modulates cytokine signaling.

In one aspect, provided herein is a reporter system for identifying a cytokine receptor modulator. Generally, the reporter system comprises at least two different fusion polypeptides and, optionally a reporter gene.

In some embodiments of any of the aspects, a first fusion polypeptide of the reporter system comprises a first domain comprising a first subunit of a multimeric receptor, e.g., a cell receptor. By a multimeric receptor is meant a receptor that is not active when in a monomeric form. However, the multimeric receptor becomes active when an agent induces two or more subunits of the receptors to form a receptor complex. It is noted that the subunits in the active receptor complex can be the same, i.e., a homomeric receptor, or the subunit of the in the active receptor complex can be different, i.e., a heteromeric receptor. In some embodiments of any one of the aspects, the multimeric receptor is a dimeric receptor.

In some embodiments of any one of the aspects, the multimeric receptor is a cytokine receptor. Cytokine receptors regulate the innate and adaptive immunity. Examples of cytokine receptors include, but are not limited to, type I cytokine receptors (e.g., IL-1R; erythropoietin receptor; GM-CSF receptor; growth hormone receptor; prolactin receptor; oncostatin M receptor; and leukemia inhibitor factor receptor); type II cytokine receptors (e.g., interferon receptors, IFNγR); immunoglobulin superfamily receptors; tumor necrosis factor receptors (TNFR); chemokine receptors; and TGF beta receptors (TGFBR). The structure and activity of cytokine receptors are known in the art and are described in detail, e.g., in Goepfert A, et al. “The human IL-17A/F heterodimer: a two-faced cytokine with unique receptor recognition properties.” Sci Rep. 2017; 7(1):8906; Moseley T A, et al. “Interleukin-17 family and IL-17 receptors.” Cytokine Growth Factor Rev. 2003; 14:155-174; Idriss H T, et al. “TNF alpha and the TNF receptor superfamily: structure-function relationship(s).”Microsc Res Tech. 2000 Aug. 1; 50(3):184-95; Schroder, K., et al. (2004), “Interferon-γ: an overview of signals, mechanisms and functions.”Journal of Leukocyte Biology, 75: 163-189; and Walter M R. “The molecular basis of IL-10 function: from receptor structure to the onset of signaling.” Curr Top Microbiol Immunol. 2014; 380:191-212. which are incorporated herein by reference in their entireties.

In some embodiments, the first domain of the first polypeptide comprises a first subunit of a cytokine receptor. For example, the first domain of the first polypeptide comprises a first subunit of a cytokine receptor selected from the group consisting of interleukin-17 receptor (IL-17R); IL-10R; interferon γ receptor (IFNγR); tumor necrosis factor receptor-1 (TNFR-1); and TNFR-2. In some embodiments, the first domain of the first polypeptide comprises a first subunit of IL-17R. For example, the first domain of the first polypeptide comprises a cytokine receptor subunit selected from the group consisting of IL-17R A subunit, (IL-17RA), IL-17R B subunit (IL-17RB) and IL-17R C subunit (IL-17RC). In some embodiments, the first domain of the first polypeptide comprises the cytokine subunit IL-17RA.

In some embodiments of any one of the aspects, the first fusion polypeptide comprises a second domain. Generally, the second domain comprises at least one fragment of a transcription activation domain and a DNA binding domain of a transcription factor.

In some embodiments of any one of the aspects, the second domain comprises a transcription factor. A transcription factor is any molecule, e.g., a polypeptide that regulate the expression of a gene. Generally, transcription factor polypeptides comprise at least one DNA-binding domain (DBD), which attaches to a specific sequence of DNA adjacent to the genes that they regulate. The transcription factors also comprise a transcription activation domain, which contains binding sites for other factors needed for transcription.

In some embodiments, the second domain of the first fusion polypeptide comprises at least a transcription activation domain and a DNA-binding domain of a transcription factor. Non-limiting examples of transcription factors that can be used include, but are not limited to, tetracycline-controlled transactivator (tTA), rtTA, LexA and VP16. In some exemplary embodiments, the second domain of the first fusion polypeptide comprises at least a transcription activation domain and a DNA-binding domain of tTA.

It is noted that the first and second domains of the first fusion polypeptide can be linked to each other in any orientation. For example, the first domain can be linked to the N-terminus of the second domain. In another non-limiting example, the first domain can be linked to the C-terminus of the second domain.

In various embodiments, the first and second domains of the first fusion polypeptide can be linked to each other via a linker. For example, the first and second domains of the first fusion polypeptide can be linked to each other via a flexible linker. Generally, the linker linking the first and second domain is a cleavable linker. For example, the cleavable linker comprises a cleavable linking group. In some embodiments, the linker comprises a cleavable linking group that is cleavable by a particular enzyme. For example, the cleavable linker comprises a comprises a protease cleavage site.

The terms “protease cleavage site” and “protease recognition site,” as interchangeably used herein, refer to a specific amino acid sequence that is recognized by a specific protease which subsequently cleaves the polypeptide by way of hydrolysis of an amide bond marked by the protease recognition site. Usually, the cleavage occurs within the recognition site. In other words, the terms “protease cleavage site” and “protease recognition site”, as interchangeably used herein, refer to a peptide sequence in the cleavable linker which can be cleaved by a selected protease thus allowing the separation of first and second domains linked by together by the linker. Exemplary protease cleavage sites include, but are not limited to, a tobacco etch virus (TEV) protease-, an Arg-C proteinase-, an Asp-N endopeptidase+N-terminal Glu-, an Asp-N endopeptidases-, an IgA-Protease-, a caspase2-,caspase3-, a caspase4-, a caspase5-, a caspase6-, a caspase7-, a caspase8-, a caspase9-, a caspasel-, a caspaselO-, a cathepsin-, a chymotrypsin-high specificity, a chymotrypsin-low specificity-, a clostripain (Clostridiopeptidase B)-, a coagulation factor protease-, a endopeptidasemannan-binding lectin-associated serine protease-, a enterokinase-SUMO Express protease-, a Factor Xa-, a furin-, a glutamyl endopeptidase-, a granzyme A-, a granzyme B-, a granzyme M-, a granzymeB-, a neurotrypsin, a pepsin-, a proline-endopeptidase-, a proteinase-, a proteinase K-, a spinesin-, a staphylococcal peptidase I-, a Thermolysin-, a Thrombin-, a tissue plasminogen activator (tPA) protease-, and a trypsin-cleavage site. In some embodiments of any one of the aspects, the linker of the first fusion polypeptide comprises a TEV protease cleavage site.

In some embodiments of any of the aspects, a second fusion polypeptide of the reporter system comprises a first domain comprising a second subunit of the multimeric receptor. For example, the first domain of the second fusion polypeptide comprises a second subunit of the multimeric receptor such that the multimeric receptor becomes active when an agent induces the first and second fusion polypeptides together form an active receptor complex. Accordingly, in some embodiments, the first domain of the second polypeptide comprises a second subunit of a cytokine receptor. For example, the first domain of the second polypeptide comprises a second subunit of a cytokine receptor selected from the group consisting of interleukin-17 receptor (IL-17R); IL-10R; interferon γ receptor (IFNγR); tumor necrosis factor receptor-1 (TNFR-1); and TNFR-2. In some embodiments, the first domain of the second polypeptide comprises a first subunit of IL-17R. For example, the first domain of the second polypeptide comprises a cytokine receptor subunit selected from the group consisting of IL-17R A subunit, (IL-17RA), IL-17R B subunit (IL-17RB) and IL-17R C subunit (IL-17RC). In some embodiments, the first domain of the second polypeptide comprises the cytokine subunit IL-17RB or IL-17RC.

In various embodiments, the second fusion polypeptide further comprises a second domain comprising at least a protease domain. As used herein, the terms “protease domain” and “catalytic domain of a protease,” as used interchangeably herein, refer to a catalytically active portion of a protease. Mentioned protease domain of the protease includes single, double and multi-chain forms of any of these proteins. The protease domain of a protein has all the necessary properties of this protein required for its proteolytic activity, such as, for example, the presence of a catalytic center. Generally, the catalytic domain of the protease is the functional fragment of the protease. As used herein, a “functional fragment” is a fragment or segment of a peptide which retains at least 50% of the wild type reference protease activity according to an assay known in the art or described below herein.

As used herein, the term “protease” refers to an enzyme that catalyzes the hydrolysis of peptide covalent bonds. These designations refer to zymogenic forms and their activated single, double and multi-chain forms. For clarity, the mention of proteases refers to all of these forms. Proteases include, for example, serine proteases, cysteine proteases, aspartic proteases, threonine and metalloproteases depending on the catalytic activity of their active center and the mechanism of cleavage of peptide bonds in the target substrate. Non-limiting examples of proteases that can be used include tobacco etch virus (TEV); thrombin; tissue plasminogen activator (tPA) serine proteases; trypsin; coagulation factor proteases; endopeptidase; neurotrypsin; chymotrypsin; mannan-binding lectin-associated serine proteases; cathepsin; furin; granzyme A; granzyme B; granzyme M; proteinase; spinesin; analogs and derivatives thereof. Additional examples of proteases and methods of generating proteases for cleavage of the transcription factor provided herein are found, e.g., International Application No. WO2004/031733A2, which is incorporated by reference in its entirety. In some embodiments of any one of the aspects, the second domain of the second fusion polypeptide comprises a protease domain of TEV.

It is noted that the first and second domains of the second fusion polypeptide can be linked to each other in any orientation. For example, the first domain can be linked to the N-terminus of the second domain. In another non-limiting example, the first domain can be linked to the C-terminus of the second domain.

In various embodiments, the first and second domains of the second fusion polypeptide can be linked to each other via a linker. For example, the first and second domains of the second fusion polypeptide can be linked to each other via a flexible linker.

As used herein, the term “linker” means a molecular moiety that connects two parts of a composition. The linker can be a chemical linker, a single peptide bond (e.g., linked directly to each other) or a peptide linker containing one or more amino acid residues (e.g. with an intervening amino acid or amino acid sequence between the two domains that are linked to each other.

In some embodiments of any one of the aspects, the linker is a flexible linker. As used herein, a “flexible linker” is a linker which does not have a fixed structure (secondary or tertiary structure) in solution and is therefore free to adopt a variety of conformations. Generally, a flexible linker has a plurality of freely rotating bonds along its backbone. In contrast, a rigid linker is a linker which adopts a relatively well-defined conformation when in solution. Rigid linkers are therefore those which have a particular secondary and/or tertiary structure in solution.

It is noted that two domains that are linked together can be separated from each other by any desired distance. In other words, the linker can be of any desired length. Inventors have discovered inter alia that certain linker lengths are relatively better for using the fusion protein in methods for detecting target nucleic acids. Accordingly, in some embodiments of the various aspects described herein, the linker is from about 10 Å to about 140 Å in length. For example, the linker can be from about 10 Å to about 130 Å in length, from about 15 Å to about 125 Å in length, from about 20 Å to about 120 Å in length, from about 25 Å to about 115 Å in length, from about 30 Å to about 110 Å in length, from about 35 Å to about 105 Å in length, from about 40 Å to about 100 Å in length, from about 45 Å to about 95 Å in length, from about 50 Å to about 90 Å in length, or from about 55 Å to about 85 Å in length. In some embodiments of the various aspects described herein, the linker can be from about 60 Å to about 80 Å in length or from about 65 Å to about 75 Å in length. In some embodiments of the various aspects described herein, the linker is about 70 Å in length.

In some embodiments of the various aspects described herein, at least two of the domains are linked via a peptide linker. The term “peptide linker” as used herein denotes a peptide with amino acid sequences, which is in some embodiments of synthetic origin. It is noted that peptide linkers may affect folding of a given fusion protein, and may also react/bind with other proteins, and these properties can be screened for by known techniques. A peptide linker can comprise 1 amino acid or more, 5 amino acids or more, 10 amino acids or more, 15 amino acids or more, 20 amino acids or more, 25 amino acids or more, 30 amino acids or more, 35 amino acids or more, 40 amino acids or more, 45 amino acids or more, 50 amino acids or more and beyond. Conversely, a peptide linker can comprise less than 50 amino acids, less than 45 amino acids, less than 40 amino acids, less than 35 amino acids, less than 30 amino acids, less than 30 amino acids, less than 25 amino acids, less than 20 amino acids, less than 15 amino acids or less than 10 amino acids.

In some embodiments of the various aspects described herein, the peptide linker comprises from about 5 amino acids to about 50 amino acids. For example, the peptide linker can comprise from about 5 amino acids to about 45 amino acids, from about 5 amino acids to about 40 amino acids, from about 5 amino acids to about 35 amino acids, from about 10 amino acids to 30 amino acids, or from about 15 amino acids to about 25 amino acids.

In some embodiments of the various aspects described herein, the linker comprises 15, 16, 17, 18, 19, 20, 21, 22, 23, 24 or 25 amino acids. For example, the linker comprises 17, 18, 19, 20, 21, 22 or 23 amino acids. Preferably, the linker comprises 18, 19, 20, 21 or 22 amino acids. More preferably, the linker comprises 19, 20 or 21 amino acids. In some embodiments of the various aspects described herein, the linker comprises 20 amino acids.

Exemplary peptide linkers include those that consist of glycine and serine residues, the so-called Gly-Ser polypeptide linkers. As used herein, the term “Gly-Ser polypeptide linker” refers to a peptide that consists of glycine and serine residues. In some embodiments of the various aspects described herein, the peptide linker comprises the amino acid sequence (GlyₓSer)ₙ (SEQ ID NO: 16), where x is 2, 3, 4 or 5, and n is 1, 2, 3, 4, 5, 6, 7, 8, 9 or 10. In some embodiments of the various aspects described herein, x is 3 and n is 3, 4, 5 or 6. In some embodiments of the various aspects described herein, x is 3 and n is 4 or 5. In some embodiments of the various aspects described herein, x is 4 and n is 3, 4, 5 or 6. In some embodiments of the various aspects described herein, x is 4 and n is 4 or 5. In some embodiments of the various aspects described herein, x is 3 and n is 2. In some embodiments of the various aspects described herein, x is 3 or 4 and n is 1.

Peptide linkers may affect folding of a given fusion protein, and may also react/bind with other proteins, and these properties can be screened for by known techniques. Exemplary linkers, in addition to those described herein, include a string of histidine residues, e.g., His6 (SEQ ID NO: 17); sequences made up of Ala and Pro, varying the number of Ala-Pro pairs to modulate the flexibility of the linker; and sequences made up of charged amino acid residues e.g., mixing Glu and Lys. Flexibility can be controlled by the types and numbers of residues in the linker. See, e.g., Perham et al., Biochem. 8501 (1991) 30: 8501 and Wriggers et al., Biopolymers 736 (2005) 80:736.

In some embodiments of the various aspects described herein, the linker can be a chemical linker. Chemical linkers can comprise a direct bond or an atom such as oxygen or sulfur, a unit such as NH, C(O), C(O)NH, SO, SO₂, SO₂NH, or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclyl, substituted or unsubstituted C₃-C₁₂ cycloalkyl, where one or more methylenes can be interrupted or terminated by O, S, S(O), SO₂, NH, or C(O).

In some embodiments of any of the aspects, the reporter system comprises a reporter gene.

As used herein, the term “reporter gene” refers to a nucleic acid that encodes a reporter molecule that can be detected by spectroscopic, photochemical, biochemical, immunochemical, electromagnetic, radiochemical, or chemical means, such as fluorescence, chemifluorescence, or chemiluminescence, or any other appropriate means. Reporter molecules generally produce a measurable signal such as fluorescence, color, or luminescence. Exemplary reporter molecules include, but are not limited to bioluminescent compounds, chromophores, antibodies, chemiluminescent compounds, fluorescent compounds, metal chelates, and enzymes.

The reporter molecule can be a protein whose presence can be readily observed. For example, fluorescent proteins fluoresce when excited with light of a particular wavelength, luciferases catalyze a reaction that produces light, and enzymes such as β-galactosidase convert a substrate to a colored product.

In some embodiments, the reporter molecule is a fluorescent protein. In other words, the reporter gene encodes a fluorescent protein. Non-limiting examples of fluorescent proteins include: a UV fluorescent protein, a blue fluorescent protein (BFP), a cyan fluorescent protein (CFP), green fluorescent protein (GFP), a yellow fluorescent protein (YFP), an orange fluorescent protein (OFP), a red fluorescent protein (RFP), a far-red fluorescent protein, a near IR fluorescent protein, a Sapphire-type fluorescent protein, or a Long Stokes shift fluorescent protein. Typically, the indicated color of the fluorescent protein is set based on its emission wavelength. Additional examples of sequences and genes encoding fluorescent proteins that can be used in accordance with the invention include, without limitation, those proteins provided in U.S. Patent Application No. 2012/0003630 (see Table 59), incorporated herein by reference in its entirety.

Examples of UV fluorescent proteins include, but are not limited to, Sirius, Sandercyanin, and shBFP-N158S/L173I. Examples of blue fluorescent proteins include, but are not limited to, Azurite, EBFP2, mKalama1, mTagBFP2, and tagBFP. Examples of cyan fluorescent proteins include, but are not limited to, ECFP, Cerulean, mCerulean3, SCFP3A, CyPet, mTurquoise, mTurquoise2, TagCFP, Mtfp1, monomeric Midoriishi-Cyan, and Aquamarine. Examples of green fluorescent proteins include, but are not limited to, TurboGFP, TagGFP2, mUKG, Superfolder GFP, Emerald, EGFP, Monomeric Azami Green, mWasabi, Clover, and mNeonGreen. Examples of yellow fluorescent proteins include, but are not limited to, TagYFP, EYFP, Topaz, Venus, SYFP2, Citrine, Ypet, IanRFP-ΔS83, and mPapaya1. Examples of orange fluorescent proteins include, but are not limited to, Monomeric Kusabira-Orange, mOrange, mOrange2, mKOκ, and Mko2. Examples of red fluorescent proteins include, but are not limited to, TagRFP, TagRFP-T, mRuby, mRuby2, mTangerine, mApple, mStrawberry, FusionRed, mCherry, and mNectarine. Examples of far red fluorescent proteins include, but are not limited to, mKate2, HcRed-Tandem, mPlum, mRaspberry, mNeptune, NirFP, TagRFP657, TagRFP675, and mCardinal. Examples of near IR fluorescent proteins include, but are not limited to, iFP1.4, iRFP713 (iRFP), iRFP670, iRFP682, iRFP702, iRFP720, and iFP2.0. Examples of sapphire-type fluorescent proteins include, but are not limited to, Sapphire, T-Sapphire, and mAmetrine. Examples of long Stokes shift fluorescent proteins include, but are not limited to, mKeima Red, mBeRFP, LSS-mKate2, LSS-mKate1, LSSmOrange, CyOFP1, and Sandercyanin.

Luciferases can also be used as reporter molecules, as cells tend to have little to no background luminescence in the absence of a luciferase. Luminescence can be readily quantified using a plate reader or luminescence counter. Examples of genes encoding luciferases that can be used in the systems described herein include, without limitation, dmMyD88-linker-Rluc, dmMyD88-linker-Rluc-linker-PEST191, Renilla luciferase, and firefly luciferase (from Photinus pyrahs).

Enzymes that produce colored substrates (“colorimetric enzymes”) can also be used as reporter molecules. Enzymatic products can be quantified using spectrophotometers or other instruments that can take absorbance measurements including plate readers. Like luciferases, enzymes such as β-galactosidase can be used for measuring low levels of gene expression because they tend to amplify low signals. Examples of genes encoding colorimetric enzymes that can be used in accordance with the systems described herein include, without limitation, lacZ alpha fragment, lacZ (encoding β-galactosidase, full-length), and xylE.

The reporter gene can be operably linked to a transcription factor regulatory element. The term “transcription factor regulatory element,” and “TFRE,” and “regulatory element,” as used herein, refer to a nucleotide sequence that is recognized and bound by a transcription factor. For example, the reporter gene can be operably linked to a transcription factor regulatory element that is recognized and bound by the DNA binding domain comprised in the second fusion polypeptide.

The reporter gene can also be operably linked to an inducible promoter. As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter.

In another aspect, provided herein is a polynucleotide encoding a fusion polypeptide provided herein. For example, the polynucleotide can encode the first fusion polypeptide and/or the second fusion polypeptide. The skilled person will understand that, due to the degeneracy of the genetic code, a given fusion polypeptide can be encoded by different polynucleotides. These “variants” are encompassed herein.

In some embodiments, the reporter system or polynucleotide provided herein encodes one or more amino acid sequences selected from the group consisting of: SEQ ID NO: 1-SEQ ID NO: 15, or a fragment thereof.

In some embodiments, a polynucleotide encoding a fusion polypeptide described herein is comprised in a vector. In some embodiments, a nucleic acid sequence encoding a fusion polypeptide is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a yeast or yeast cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

The disclosure also provides a cell comprising a fusion polypeptide described herein or a polynucleotide encoding the same. As used herein, the term “cell” refers to a single cell as well as to a population of (i.e., more than one) cells. For example, a cell prokaryotic cell or a eukaryotic cell comprising a polypeptide or polynucleotide described herein. Exemplary cells include, but are not limited to, bacterial cells, yeast cells, plant cell, animal (including insect) or human cells. In some embodiments the cell selected from hepatocytes, fibroblasts, neurons, adipocytes, kidney and immune cells. In some embodiments, the cell is a Th17 cell.

The first and the second fusion polypeptides provided herein can form a heterotrimeric receptor complex upon binding with an agent. Formation of the heterotrimeric receptor complex can induce expression of the reporter gene, thereby forming the reporter molecule. Thus, the reporter system described herein can be used for finding agents that induce formation of receptor complex. In other words, the reporter system provided herein can be used to screen for a desired activity and/or property of an agent. The method of screening comprises contacting the reporter system or a cell expressing said reporter system provided herein with a test agent. The test agent, for example, can be selected from a library of diverse compounds.

In some embodiments, the reporter system described herein can be used for identifying test agents that modulate an immune response. Generally, the method comprises contacting a test agent with a cell comprising a reporter system provided herein and detecting or measuring an expression level of the reporter gene, i.e., measuring a level of the reporter molecule. The expression level of the reporter can be compared to a control or reference level. A change in the expression level of the reporter gene relative to the control or reference level indicates the agent modulates an immune response. For example, an increase in the expression level of the reporter gene relative to a control or reference level means that the agent modulates an immune response.

As such, the reporter system described herein can be used for identifying cytokine receptor modulators. A cytokine receptor modulator is a compound, ligand, agent, agonist, antagonist, or therapeutic that binds to and/or interacts with the reporter system provided herein and promotes downstream effector signaling by the reporter system to induce expression of the reporter gene provided herein.

Exemplary embodiments of the disclosure can be described by the following numbered embodiments:

Embodiment 1: A reporter system for identifying a cytokine receptor modulator, the system comprising: (a) a first fusion polypeptide comprising: (i) a first domain comprising a first subunit of a cytokine receptor; (ii) a second domain comprising at least a transcription activation domain and a DNA-binding domain of a transcription factor; and (iii) a linker linking the first and second domain, wherein the linker comprises a protease cleavage site between the first and second domain; and (b) a second fusion polypeptide comprising: (i) a first domain comprising a second subunit of the cytokine receptor; (ii) a second domain comprising at least a catalytic domain of a protease; (iii) and a linker linking the first and second domain; and (c) a reporter gene operably linked to a transcription factor regulatory element (TFRE), wherein the DNA-binding domain of a transcription factor recognizes and binds the TFRE.

Embodiment 2: The reporter system of Embodiment 1, wherein the linker linking the first and second domain of the first fusion polypeptide is a flexible linker.

Embodiment 3: The reporter system of Embodiment 1 or 2, wherein the linker linking the first and second domain of the second fusion polypeptide is a flexible linker.

Embodiment 4: The reporter system of any one of Embodiments 1-3, wherein the cytokine receptor is selected from the group consisting of: interleukin-17 receptor (IL-17R); IL-10R; interferon γ receptor (IFNγR); tumor necrosis factor receptor-1 (TNFR-1); and TNFR-2.

Embodiment 5: The reporter system any one of Embodiments 1-4, wherein the first and second subunit of the cytokine receptor are selected from IL-17R A subunit, (IL-17RA), IL-17R B subunit (IL-17RB) and IL-17R C subunit (IL-17RC).

Embodiment 6: The reporter system of any one of Embodiments 1-5, wherein the first subunit of the cytokine receptor is IL-17RA.

Embodiment 7: The receptor system of any one of Embodiments 1-6, wherein the second subunit of the cytokine receptor is IL-17RB or IL-17RC.

Embodiment 8: The reporter system of any one of Embodiments 1-5, wherein the first subunit of the cytokine receptor is IL-17RB or IL-17RC.

Embodiment 9: The receptor system of any one of Embodiments 1-5 or 8, wherein the second cytokine receptor domain IL-17RA.

Embodiment 10: The reporter system of any one of Embodiments 1-9, wherein the transcription factor is selected from the group consisting of: tetracycline-controlled transactivator (tTA), rtTA, LexA and VP16.

Embodiment 11: The reporter system of any one of Embodiments 1-10, wherein the transcription factor is tTA.

Embodiment 12: The reporter system of any one of Embodiments 1-11, wherein the protease is selected from the group consisting of: tobacco etch virus (TEV) protease and thrombin.

Embodiment 13: The reporter system of any one of Embodiments 1-12, wherein the protease is a tobacco etch virus (TEV) protease.

Embodiment 14: The reporter system of any one of Embodiments 1-13, wherein the first subunit of a cytokine receptor is IL-17RA and the transcription factor is tTA.

Embodiment 15: The T reporter system of any one of Embodiments 1-14, wherein the first subunit of a cytokine receptor is IL-17RB or IL17RC and the protease is TEV.

Embodiment 16: The reporter system of any one of Embodiments 1-15, wherein the reporter gene is operably linked to an inducible promotor.

Embodiment 17: The reporter system of any one of Embodiments 1-16, wherein the reporter gene is selected from the group consisting of: green fluorescent protein (GFP), cyan fluorescent protein (CFP), red fluorescent protein (RFP), β-galactosidase (lacZ), chloramphenicol acetyltransferase (cat), and luciferase (luc).

Embodiment 18: A cell comprising the reporter system of any one of Embodiments 1-17.

Embodiment 19: A polynucleotide encoding at least two of the first fusion polypeptide, the second fusion polypeptide and the reporter gene of any one of Embodiments 1-17.

Embodiment 20: A cell comprising the polynucleotide of Embodiment 19.

Embodiment 21: The cell of Embodiment 18 or 20, wherein the cell is selected from the group consisting of hepatocytes, fibroblasts, neurons, adipocytes, kidney cells and immune cells.

Embodiment 22: A method for identifying a test agent that modulates an immune response, the method comprising: (i) contacting a cell with a test agent, wherein the cell comprises a reporter system of any one of Embodiments 1-17; and (ii) detecting an expression level of the reporter gene, wherein an increase in the expression level of the reporter gene relative to a control or reference level indicates the agent modulates an immune response.

The method of Embodiment 22, wherein said detecting the expression level of the reporter gene comprises spectroscopic, photochemical, biochemical, immunochemical, electrical, optical and/or chemical detection.

The method of Embodiment 22 or 23, wherein said detecting the expression level of the reporter gene comprises fluorescence detection, luminescence detection, chemilluminescence detection, immunofluorescence detection or FRET detection.

The method of any one of Embodiments 22-23, wherein the test agent is selected from the group consisting of small molecules, peptides, polypeptides, oligonucleotides, and polynucleotides, and lipids.

### Some Selected Definitions:

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “fusion polypeptide” refers to an engineered polypeptide or protein that is linked to another polypeptide. For example, the term can refer to, but is not limited to, a fusion polypeptide comprising a first domain comprising a first subunit of a cytokine receptor; a second domain comprising a transcription activation domain and a DNA-binding domain of a transcription factor; and a linker linking the first and second domain.

As used herein, an “agent” or “test agent” refers to any compound or substance such as, but not limited to, a small molecule, nucleic acid, polypeptide, peptide, drug, ion, etc. An “agent” can be any chemical, entity or moiety, including without limitation, synthetic and naturally-occurring proteinaceous and non-proteinaceous entities. In some embodiments of any of the aspects, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants. In some embodiments, the agent is a ligand of IL-17R, TNFR, or IL-10R or a derivative thereof. In some embodiments, the agent modulates the activity of IL-17a or IL-17Ra.

As used herein, the term “small molecule” refers to a organic or inorganic molecule, either natural (i.e., found in nature) or non-natural (i.e., not found in nature), which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Examples of “small molecules” that occur in nature include, but are not limited to, taxol, dynemicin, and rapamycin. Examples of “small molecules” that are synthesized in the laboratory include, but are not limited to, compounds described in Tan et al., (“Stereoselective Synthesis of over Two Million Compounds Having Structural Features Both Reminiscent of Natural Products and Compatible with Miniaturized Cell-Based Assays” J. Am. Chem. Soc. 120:8565, 1998; incorporated herein by reference). In some embodiments, natural-product-like small molecules are utilized in the screen provided herein.

The term “gene” means a nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “contacting” when used in reference to a cell, encompasses subjecting the cells to an appropriate culture media, which comprises the test agent.

As used herein, the term “modulates” refers to an effect including increasing or decreasing a given parameter as those terms are defined herein.

The terms “decrease”, “reduced”, “reduction”, or “inhibit” are all used herein to mean a decrease or lessening of a property, level, or other parameter by a statistically significant amount.

In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, a “reference level” or “control level” refers to the expression level of the reporter gene in absence of the test agent.

As used herein, the term “inflammation” or “inflamed” refers to activation or recruitment of the immune system or immune cells (e.g., T cells, B cells, macrophages). A tissue that has inflammation can become reddened, white, swollen, hot, painful, exhibit a loss of function, or have a film or mucus. Immune cells may secrete cytokines and interferons to signal other immune cells and promote phagocytosis of the microorganism and infected cells. Methods of identifying inflammation are well known in the art. Inflammation typically occurs following injury or infection by a microorganism. Inflammation can result in the release of cytokines by T cells. These cytokines are known to have various effects on the immune response and target tissues (e.g. the brain).

As used herein, the term “cytokine” refers to a small protein (˜5-20 kDa) that acts through a target cytokine receptor to modulate the immune response, cell growth, or other cellular functions.

As used herein, the term “interleukin-17 receptor,” refer to an interleukin-17 receptor that is expressed in the brain, hematopoietic, bone marrow, thymus, spleen, intestine, and lung, among many others. Specifically, IL-17R can regulate numerous cell-specific functions. For example, IL-17Ra is involved in cell signaling events related to brain activity, inflammation, and the like. Sequences for IL-17Ra, are known for a number of species, e.g., human IL-17RA (NCBI GeneID: 23765) polypeptide and mRNA (e.g., NCBI Reference Sequences: NM_014339.6; NM_001289905.1; NP_055154.3; and NP_001276834.1). IL-17Ra can refer to human IL-17R (e.g., IL-17Ra), including naturally occurring variants, molecules, genetically engineered IL-17Ra, and alleles thereof. IL-17Ra refers to the mammalian IL-17Ra of, e.g., mouse, rat, rabbit, dog, cat, cow, horse, pig, and the like. The amino acid sequence of human IL-17R is shown in SEQ ID NO: 2 for isoform 1 and SEQ ID NO: 3 for isoform 2.

As used herein, the term “interferon gamma” or “IFNγ,” or “IFNG” refers to a dimerized soluble cytokine (interferon) that is responsible for immune responses in the body and activates interferon gamma receptors (IFNGR1 and IFNGR2). Specifically, IFNγ can regulate numerous cell-specific functions. For example, as described herein, IFNγ is involved in cell signaling events related to blood brain barrier permeability, inflammation, and the like. Sequences for IFNγ, are known for a number of species, e.g., human IFNG (NCBI GeneID: 3458) polypeptide and mRNA (e.g., NCBI Reference Sequences: NP_000610.2 and NM_000619.3). IFNγ can refer to human IFNγ, including naturally occurring variants, molecules, genetically engineered IFNγ, and alleles thereof. IFNγ refers to the mammalian IFNγ of, e.g., mouse, rat, rabbit, dog, cat, cow, horse, pig, and the like. The amino acid sequence of human IFNγ is shown in SEQ ID NO: 4.

The term “statistically significant” or “significantly” refers to statistical significance and generally means a two standard deviation (2SD) or greater difference.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin Exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

Unless otherwise explained, all technical and scientific terms used herein have the same meaning as commonly understood by one of ordinary skill in the art to which this disclosure belongs.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The present invention is further illustrated by the following Examples, which in no way should be construed as further limiting.

### EXAMPLE

### Example 1: Novel Screening Platform to Identify Immune Modulatory Agents

Immune cells often exert their effects by secreting cytokines. Cytokines then bind to their cognate receptors expressed on the membranes of target cells, binding of which initiates a downstream signaling cascade leading to various biological effects. For example, T helper (Th)17 cells, which are responsible for mounting immune responses against extracellular pathogens and fungi, mediate their effects by producing cytokines including interleukin-17a (IL-17a). Under pathological conditions, however, Th17 cells and IL-17a promote inflammatory and autoimmune diseases¹. Genome-wide association studies in humans have linked genes involved in Th17 cell differentiation and function (e.g., IL23R) with susceptibility to Crohn's disease, arthritis, and psoriasis²⁻⁴. Consistent with these, blocking antibodies targeting IL-17a or its receptor (IL-17R) have also been developed, resulting in successful clinical trials with psoriatic patients⁵. Based on these results, the FDA approved an anti-IL-17a antibody (e.g. secukinumab) for the treatment of moderate-to-severe plaque psoriasis. Our recent data suggest that Th17 cells also function as critical mediators, working in pregnant mice, to induce neurodevelopmental disorder-like phenotypes in offspring prenatally exposed to maternal immune activation (MIA)⁶. These MIA-associated neurodevelopmental phenotypes are mediated by activation of IL-17R in the developing fetal brain⁷. Therefore, offspring are protected from developing abnormal behavioral and cortical phenotypes by either selective removing Th17 cell population or inhibiting IL-17a activity. While increased IL-17R activity during embryogenesis promotes behavioral abnormalities, our recent data suggested that increasing IL-17R activity in adulthood of MIA offspring leads to amelioration of behavioral symptoms, social behavioral phenotypes.

Therefore, modulating IL-17R activity with small molecules may have therapeutic value not only in controlling autoimmunity but also in preventing and/or therapeutically treating certain types of neurodevelopmental disorders. Until now, however, what have been developed are mostly blocking antibodies, whose main effects are to suppress activities of cytokines and their receptors. Small molecule modulators will be advantageous as they can modulate cytokine-receptor activity bi-directionally. They also can be administered orally unlike protein biologics and they are generally more stable.

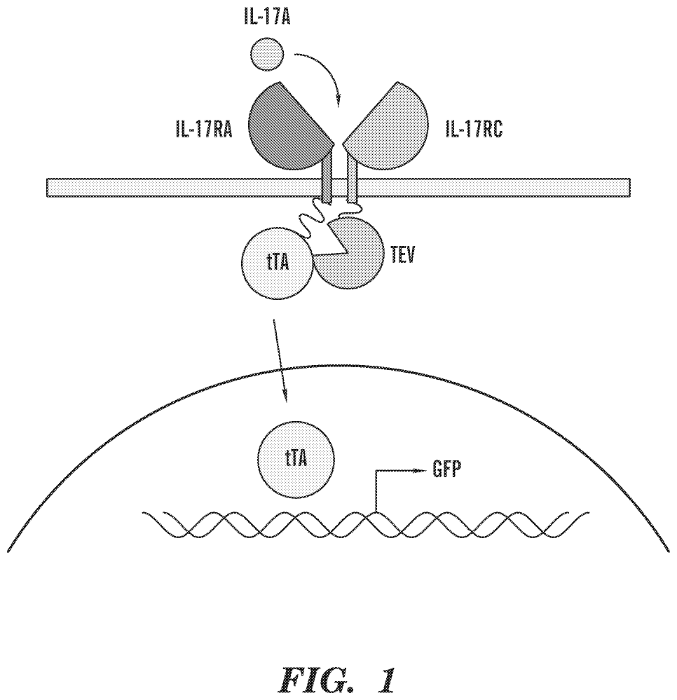

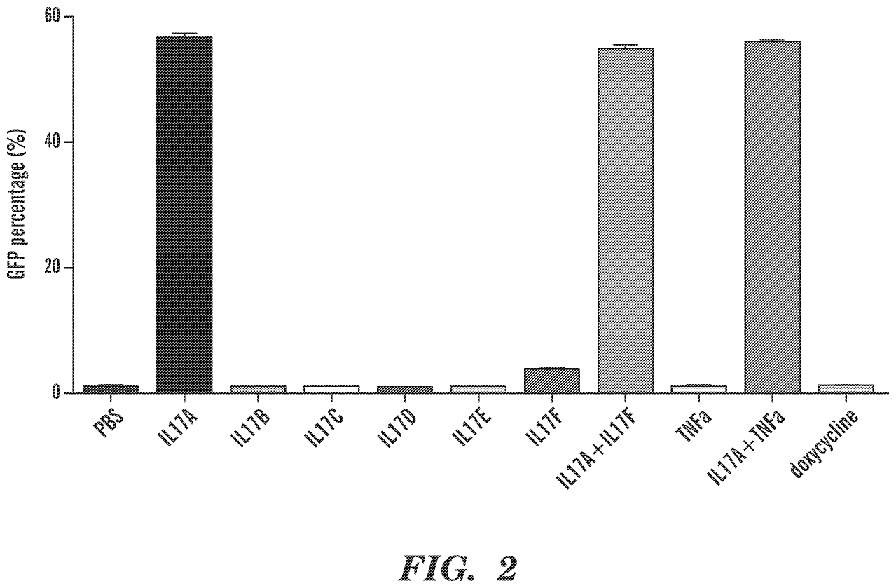

To identify small molecule compounds that control IL-17R activity, inventors developed a novel screening platform that can be used to monitor IL-17a binding to its receptor. To the best of inventors' knowledge, no such reporter system has been developed for any cytokine-cytokine receptor and not just for the IL-17a/IL-17R. Upon IL-17a binding to its receptor subunit A (IL-17RA), another receptor subunit IL-17RC is recruited, and these two subunits form a heterodimer. In the exemplary reporter system, a transcription factor (tTA) was tethered, via a protease cleavage site, to IL-17RA. In addition, a tobacco etch virus (TEV) protease was fused to the receptor subunit IL-17RC. Upon the IL-17a-dependent recruitment of IL-17RC-TEV to IL-17RA-tTA, the tTA is cleaved off from its membrane anchor IL-17RA. The released tTA then enters the nucleus and activates the reporter gene (in this case, GFP) (FIG. 1). As shown in FIG. 2, the IL-17RA/RC reporter system responds highly selectively to IL-17a, but not to other related IL-17 family cytokines (except IL-17f).

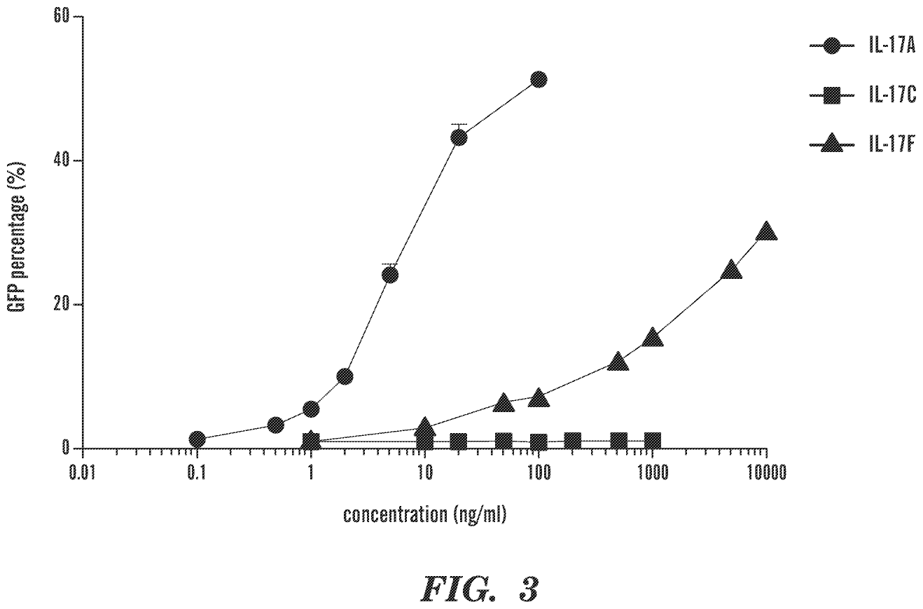

Furthermore, the reporter gene (GFP) is activated in a dose-dependent manner by IL-17a or (to a lesser degree) IL-17f, but not by IL-17c (FIG. 3). Thus, the inventors successfully generated a novel screening platform with a high signal-to-background ratio and specificity that can be used to identify small molecule and/or protein modulators that enhance or suppress IL-17a and its receptor binding.

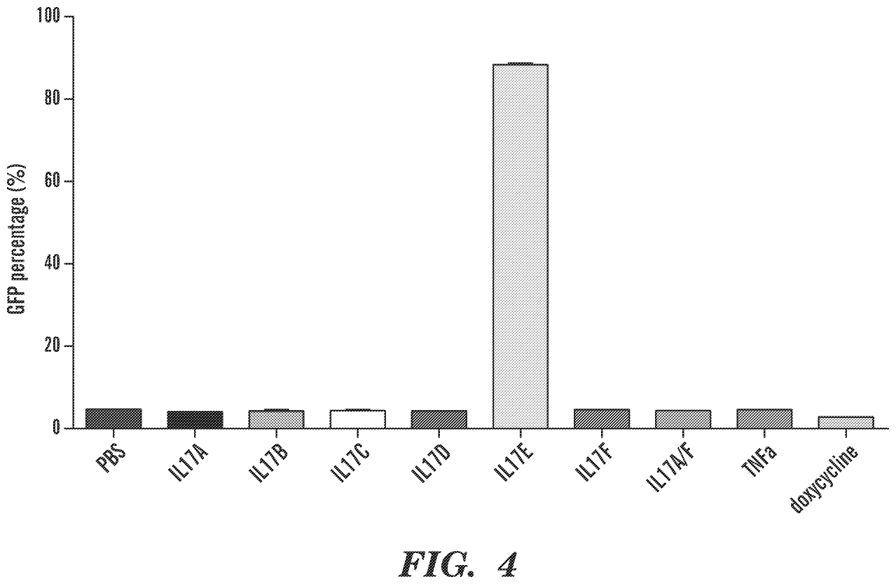

Using a similar approach, the inventors have recently also successfully generated the IL-17RA/IL-17RB reporter line (FIG. 4). The IL-17RA/RB reporter system responds highly to IL-17E (the natural ligand of IL-17RA/RB), but not to other IL-17 family proteins. Since IL-17E plays a role in allergy and asthma, small molecules modulators that will be identified using this platform may create a new IP opportunity.

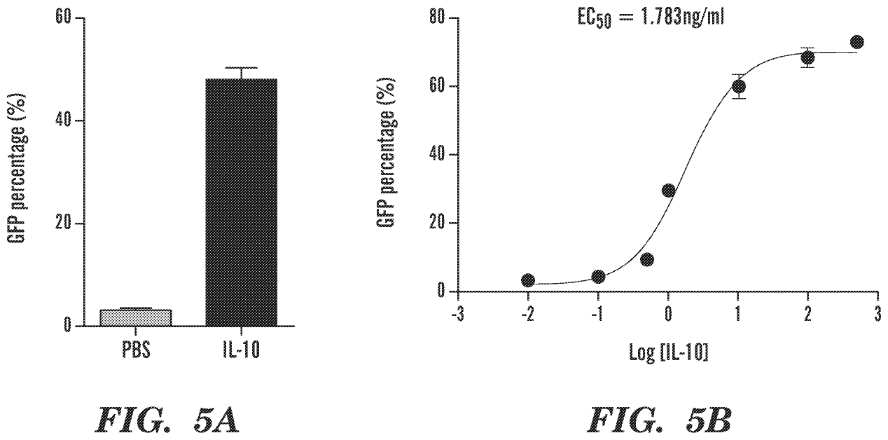

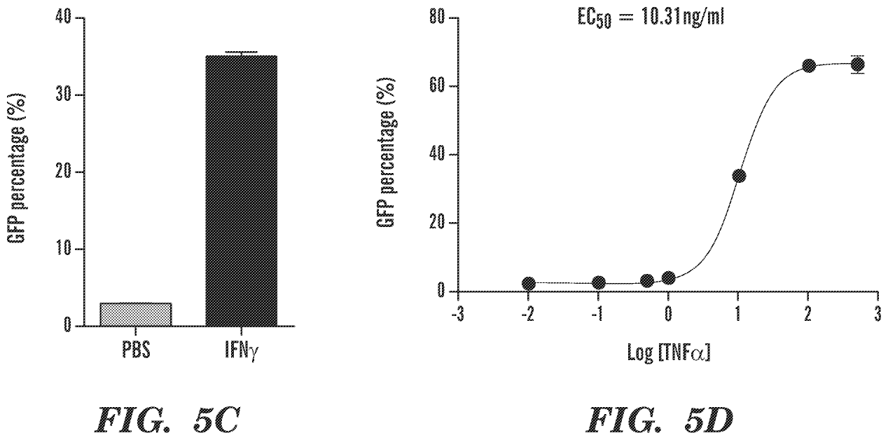

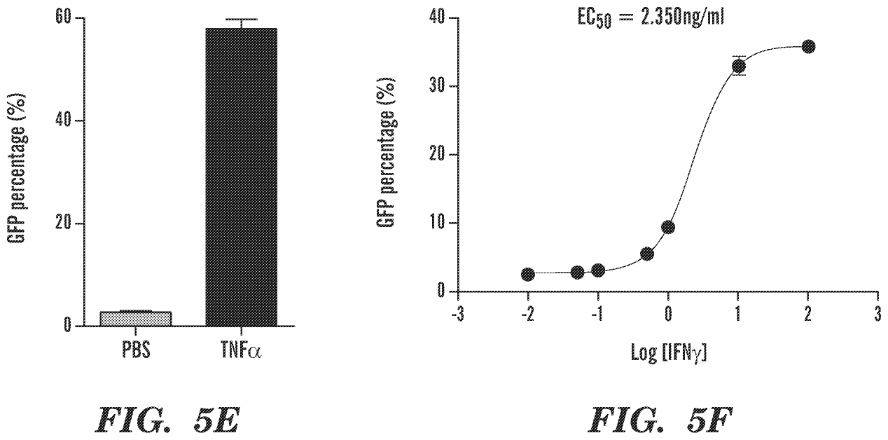

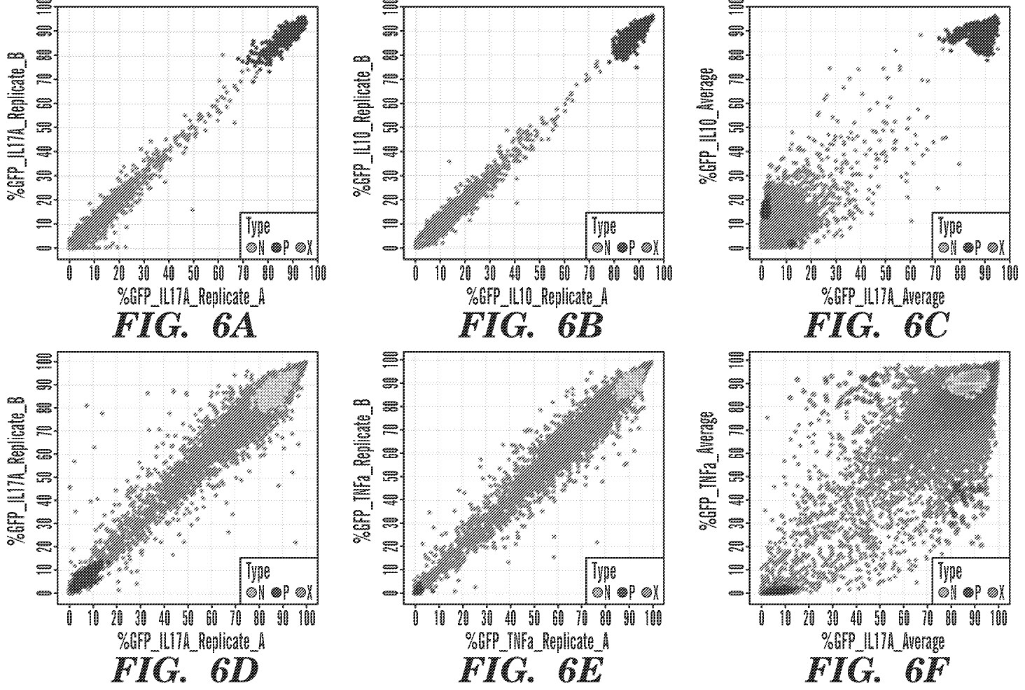

Based on these data, the following exemplary reporter systems are generated: (1) IL-17RA/IL-17RC reporter system and (2) a IL-17RA/IL-17RB reporter system. Furthermore, the systems and methods provided herein are applied to generate other cytokine/cytokine receptor reporter system (tTA fused onto one receptor subunit via a protease cleavage site, TEV fused to another receptor subunit, optimal linkers inserted between different proteins to ensure high signal-to-background ratio). The screening platform provided herein are used to identify immune modulatory agents including, but not limited to, small molecules and proteins that modulate cytokine signaling (FIGS. 5A-6F).

### REFERENCES

1 Wilke, C. M., Bishop, K., Fox, D. & Zou, W. Deciphering the role of Th17 cells in human disease. Trends Immunol 32, 603-611, doi:S1471-4906(11)00142-6 [pii] 10.1016/j.it.2011.08.003 (2011).
2 Duerr, R. H. et al. A genome-wide association study identifies IL23R as an inflammatory bowel disease gene. Science 314, 1461-1463, doi:1135245 [pii] 10.1126/science.1135245 (2006).
3 Nair, R. P. et al. Genome-wide scan reveals association of psoriasis with IL-23 and NF-kappaB pathways. Nat Genet 41, 199-204, doi:ng.311 [pii] 10.1038/ng.311 (2009).
4 Stahl, E. A. et al. Genome-wide association study meta-analysis identifies seven new rheumatoid arthritis risk loci. Nat Genet 42, 508-514, doi:ng.582 [pii] 10.1038/ng.582 (2010).
5 Miossec, P. & Kolls, J. K. Targeting IL-17 and TH17 cells in chronic inflammation. Nature reviews. Drug discovery 11, 763-776, doi:10.1038/nrd3794 (2012).
6 Choi, G. B. et al. The maternal interleukin-17a pathway in mice promotes autism-like phenotypes in offspring. Science 351, 933-939, doi:10.1126/science.aad0314 (2016).
7 Shin Yim, Y. et al. Reversing behavioural abnormalities in mice exposed to maternal inflammation. Nature 549, 482-487, doi:10.1038/nature23909 (2017).

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### SEQUENCES

SEQ ID NO: 1 (interleukin-17A precursor [Homo sapiens]; NCBI Reference Sequence:
NP_002181.1):
mtpgktslvs lllllsleai vkagitiprn pgcpnsedkn fprtvmvnln ihnrntntnp krssdyynrs tspwnlhrne
dperypsviw eakcrhlgci nadgnvdyhm nsvpiqqeil vlrrepphcp nsfrlekilv svgctcvtpi vhhva
SEQ ID NO: 2 (interleukin-17 receptor A isoform 1 precursor [Homo sapiens]; NCBI
Reference Sequence: NP_055154.3):
mgaarsppsa vpgpllglll lllgvlapgg aslrlldhra lvcsqpglnc tvknstcldd swihprnltp sspkdlqiql
hfahtqqgdl
fpvahiewtl qtdasilyle gaelsvlqln tnerlcvrfe flsklrhhhr rwrftfshfv vdpdqeyevt vhhlpkpipd
gdpnhqsknf lvpdceharm kvttpcmssg slwdpnitve tleahqlrvs ftlwnesthy qilltsfphm enhscfehmh
hipaprpeef hqrsnvtltl rnlkgccrhq vqiqpffssc lndclrhsat vscpempdtp epipdymplw vywfitgisi
llvgsvilli vcmtwrlagp gsekysddtk ytdglpaadl ippplkprkv wiiysadhpl yvdvvlkfaq flltacgtev
aldlleeqai seagvmtwvg rqkqemvesn skiivlesrg trakwqallg rgapvrlred hgkpvgdlft aamnmilpdf
krpacfgtyv vcyfsevscd gdvpdlfgaa pryplmdrfe evyfriqdle mfqpgrmhrv gelsgdnylr spggrqlraa
ldrfrdwqvr cpdwfecenl ysaddqdaps ldeevfeepl lppgtgivkr aplvrepgsq aclaidplvg eeggaavakl
ephlqprgqp apqplhtlvl aaeegalvaa vepgpladga avrlalageg eacpllgspg agrnsvlflp vdpedsplgs
stpmaspdll pedvrehleg lmlslfeqsl scqaqggcsr pamvltdpht pyeeeqrqsv qsdqgyisrs spqppeglte
meeeeeeeqd pgkpalplsp edleslrslq rqllfrqlqk nsgwdtmgse segpsa
SEQ ID NO: 3 (interleukin-17 receptor A isoform 2 precursor [Homo sapiens];
NCBI Reference Sequence: NP_001276834.1)
mgaarsppsa vpgpllglll lllgvlapgg aslrlldhra lvcsqpglnc tvknstcldd swihprnltp sspkdlqiql
hfahtqqgdl fpvahiewtl qtdasilyle gaelsvlqln tnerlcvrfe flsklrhhhr rwrftfshfv vdpdqeyevt
vhhlpkpipd
gdpnhqsknf lvpdceharm kvttpcmssg slwdpnitve tleahqlrvs ftlwnesthy qilltsfphmenhscfehmh
hipaprpeef hqrsnvtltl rnlkgccrhq vqiqpffssc lndclrhsat vscpempdtp epipgpgsek ysddtkytdg
lpaadlippp lkprkvwiiy sadhplyvdv vlkfaqfllt acgtevaldl leeqaiseag vmtwvgrqkq emvesnskii
vlcsrgtrak wqallgrgap vrlrcdhgkp vgdlftaamn milpdfkrpa cfgtyvvcyf sevscdgdvp dlfgaapryp
lmdrfeevyf riqdlemfqp grmhrvgels gdnylrspgg rqlraaldrf rdwqvrcpdw fecenlysad dqdapsldee
vfeepllppg tgivkraplv repgsqacla idplvgeegg aavaklephl qprgqpapqp lhtlvlaaee galvaavepg
pladgaavrl alagegeacp llgspgagrn svlflpvdpe dsplgsstpm aspdllpedv rehleglmls lfeqslscqa
qggcsrpamv ltdphtpyee eqrqsvqsdq gyisrsspqp pegltemeee eeeeqdpgkp alplspedle slrslqrqll
frqlqknsgw dtmgsesegp sa
SEQ ID NO: 4 (interferon gamma precursor [Homo sapiens]); NCBI Reference
Sequence: NP_000610.2):
mkytsyilaf qlcivlgslg cycqdpyvke aenlkkyfna ghsdvadngt lflgilknwk eesdrkimqs qivsfyfklf
knfkddqsiq ksvetikedm nvkffnsnkk krddfekltn ysvtdlnvqr kaiheliqvm aelspaaktg krkrsqmlfr
grrasq
SEQ ID NO: 5 (IL17RA-linker-tTA (Mus musculus)):
MAIRRCWPRVVPGPALGWLLLLLNVLAPGRASPRLLDFPAPVCAQEGLSCRVKNSTCLD
DSWIHPKNLTPSSPKNIYINLSVSSTQHGELVPVLHVEWTLQTDASILYLEGAELSVLQLN
TNERLCVKFQFLSMLQHHRKRWRFSFSHFVVDPGQEYEVTVHHLPKPIPDGDPNHKSKII
FVPDCEDSKMKMTTSCVSSGSLWDPNITVETLDTQHLRVDFTLWNESTPYQVLLESFSDS
ENHSCFDVVKQIFAPRQEEFHQRANVTFTLSKFHWCCHHHVQVQPFFSSCLNDCLRHAV
TVPCPVISNTTVPKPVADYIPLWVYGLITLIAILLVGSVIVLIICMTWRLSGADQEKHGLGS
ENLYFQYRLDKSKVINSALELLNEVGIEGLTTRKLAQKLGVEQPTLYWHVKNKRALLDA
LAIEMLDRHHTHFCPLEGESWQDFLRNNAKSFRCALLSHRDGAKVHLGTRPTEKQYETL
ENQLAFLCQQGFSLENALYALSAVGHFTLGCVLEDQEHQVAKEERETPTTDSMPPLLRQ
AIELFDHQGAEPAFLFGLELIICGLEKQLKCESGSAYSRARTKNNYGSTIEGLLDLPDDDA
PEEAGLAAPRLSFLPAGHTRRLSTAPPTDVSLGDELHLDGEDVAMAHADALDDFDLDML
GDGDSPGPGFTPHDSAPYGALDMADFEFEQMFTDALGIDEYGG
SEQ ID NO: 6 (IFNγR1-linker-tTA (Mus musculus)):
MGPQAAAGRMILLVVLMLSAKVGSGALTSTEDPEPPSVPVPTNVLIKSYNLNPVVCWEY
QNMSQTPIFTVQVKVYSGSWTDSCTNISDHCCNIYEQIMYPDVSAWARVKAKVGQKES
DYARSKEFLMCLKGKVGPPGLEIRRKKEEQLSVLVFHPEVVVNGESQGTMFGDGSTCYT
FDYTVYVEHNRSGEILHTKHTVEKEECNETLCELNISVSTLDSRYCISVDGISSFWQVRTE
KSKDVCIPPFHDDRKDSIWILVVAPLTVFTVVILVFAYWYTKKNSFKRKSIMLLGSENLY
FQYRLDKSKVINSALELLNEVGIEGLTTRKLAQKLGVEQPTLYWHVKNKRALLDALAIE
MLDRHHTHFCPLEGESWQDFLRNNAKSFRCALLSHRDGAKVHLGTRPTEKQYETLENQ
LAFLCQQGFSLENALYALSAVGHFTLGCVLEDQEHQVAKEERETPTTDSMPPLLRQAIEL
FDHQGAEPAFLFGLELIICGLEKQLKCESGSAYSRARTKNNYGSTIEGLLDLPDDDAPEEA
GLAAPRLSFLPAGHTRRLSTAPPTDVSLGDELHLDGEDVAMAHADALDDFDLDMLGDG
DSPGPGFTPHDSAPYGALDMADFEFEQMFTDALGIDEYGG
SEQ ID NO: 7 (IL10RA-linker-tTA (Mus musculus)):
MLSRLLPFLVTISSLSLEFIAYGTELPSPSYVWFEARFFQHILHWKPIPNQSESTYYEVALK
QYGNSTWNDIHICRKAQALSCDLTTFTLDLYHRSYGYRARVRAVDNSQYSNWTTTETRF
TVDEVILTVDSVTLKAMDGIIYGTIHPPRPTITPAGDEYEQVFKDLRVYKISIRKFSELKNA
TKRVKQETFTLTVPIGVRKFCVKVLPRLESRINKAEWSEEQCLLITTEQYFTVTNLSILVIS
MLLFCGILVCLVLQWYIRHPGKLPTVLVFKKPLGSENLYFQYRLDKSKVINSALELLNEV
GIEGLTTRKLAQKLGVEQPTLYWHVKNKRALLDALAIEMLDRHHTHFCPLEGESWQDF
LRNNAKSFRCALLSHRDGAKVHLGTRPTEKQYETLENQLAFLCQQGFSLENALYALSAV
GHFTLGCVLEDQEHQVAKEERETPTTDSMPPLLRQAIELFDHQGAEPAFLFGLELIICGLE
KQLKCESGSAYSRARTKNNYGSTIEGLLDLPDDDAPEEAGLAAPRLSFLPAGHTRRLSTA
PPTDVSLGDELHLDGEDVAMAHADALDDFDLDMLGDGDSPGPGFTPHDSAPYGALDM
ADFEFEQMFTDALGIDEYGG
SEQ ID NO: 8 (TNFαR1-linker-tTA (Homo sapiens)):
MGLSTVPDLLLPLVLLELLVGIYPSGVIGLVPHLGDREKRDSVCPQGKYIHPQNNSICCTK
CHKGTYLYNDCPGPGQDTDCRECESGSFTASENHLRHCLSCSKCRKEMGQVEISSCTVD
RDTVCGCRKNQYRHYWSENLFQCFNCSLCLNGTVHLSCQEKQNTVCTCHAGFFLRENE
CVSCSNCKKSLECTKLCLPQIENVKGTEDSGTTVLLPLVIFFGLCLLSLLFIGLMYRYQRW
KSKLYSIVCGLGSENLYFQLRLDKSKVINSALELLNEVGIEGLTTRKLAQKLGVEQPTLY
WHVKNKRALLDALAIEMLDRHHTHFCPLEGESWQDFLRNNAKSFRCALLSHRDGAKVH
LGTRPTEKQYETLENQLAFLCQQGFSLENALYALSAVGHFTLGCVLEDQEHQVAKEERE
TPTTDSMPPLLRQAIELFDHQGAEPAFLFGLELIICGLEKQLKCESGSAYSRARTKNNYGS
TIEGLLDLPDDDAPEEAGLAAPRLSFLPAGHTRRLSTAPPTDVSLGDELHLDGEDVAMAH
ADALDDFDLDMLGDGDSPGPGFTPHDSAPYGALDMADFEFEQMFTDALGIDEYGG
SEQ ID NO: 9 (IL17RC-linker-TEV (Mus musculus)):
MPVSWFLLSLALGRNPVVVSLERLMEPQDTARCSLGLSCHLWDGDVLCLPGSLQSAPGP
VLVPTRLQTELVLRCPQKTDCALCVRVVVHLAVHGHWAEPEEAGKSDSELQESRNASL
QAQVVLSFQAYPIARCALLEVQVPADLVQPGQSVGSAVEDCFEASLGAEVQIWSYTKPR
YQKELNLTQQLPDCRGLEVRDSIQSCWVLPWLNVSTDGDNVLLTLDVSEEQDFSFLLYL
RPVPDALKSLWYKNLTGPQNITLNHTDLVPCLCIQVWSLEPDSERVEFCPFREDPGAHRN
LWHIARLRVLSPGVWQLDAPCCLPGKVTLCWQAPDQSPCQPLVPPVPQKNATVNEPQD
FQLVAGHPNLCVQVSTWEKVQLQACLWADSLGPFKDDMLLVEMKTGLNNTSVCALEP
SGCTPLPSMASTRAARLGEELLQDFRSHQCMQLWNDDNMGSLWACPMDKYIHRRWVL
VWLACLLLAAALFFFLLLKKDRRKAARGSRTALGSSLFKGPRDYNPISSTICHLTNESDG
HTTSLYGIGFGPFIITNKHLFRRNNGTLLVQSLHGVFKVKNTTTLQQHLIDGRDMIIIRMP
KDFPPFPQKLKFREPQREERICLVTTNFQTKSMSSMVSDTSCTFPSSDGIFWKHWIQTKDG
QCGSPLVSTRDGFIVGIHSASNFTNTNNYFTSVPKNFMELLTSQEAQQWVSGWRLNADS
VLWGGHKVFMSKPEEPFQPVKEATQLMNELVYSYPYDVPDYA
SEQ ID NO: 10 (IL17RB-linker-TEV (Mus musculus)):
MLLVLLILAASCRSALPREPTIQCGSETGPSPEWMVQHTLTPGDLRDLQVELVKTSVAAE
EFSILMNISWILRADASIRLLKATKICVSGKNNMNSYSCVRCNYTEAFQSQTRPSGGKWT
FSYVGFPVELSTLYLISAHNIPNANMNEDSPSLSVNFTSPGCLNHVMKYKKQCTEAGSLW
DPDITACKKNEKMVEVNFTTNPLGNRYTILIQRDTTLGFSRVLENKLMRTSVAIPVTEESE
GAVVLTPYLHTCGNDCIRREGTVVLCSETSAPIPPDDNRRMLGGWLPLFLVLLVAVWV
LAAGIYLTWRQGRSTKTSFPISGSSLFKGPRDYNPISSTICHLTNESDGHTTSLYGIGFGPFII
TNKHLFRRNNGTLLVQSLHGVFKVKNTTTLQQHLIDGRDMIIIRMPKDFPPFPQKLKFRE
PQREERICLVTTNFQTKSMSSMVSDTSCTFPSSDGIFWKHWIQTKDGQCGSPLVSTRDGFI
VGIHSASNFTNTNNYFTSVPKNFMELLTSQEAQQWVSGWRLNADSVLWGGHKVFMSKP
EEPFQPVKEATQLMNELVYSYPYDVPDYA
SEQ ID NO: 11 (IFNγR2-linker-TEV (Mus musculus)):
MRPLPLWLPSLLLCGLGAAASSPDSFSQLAAPLNPRLHLYNDEQILTWEPSPSSNDPRPVV
YQVEYSFIDGSWHRLLEPNCTDITETKCDLTGGGRLKLFPHPFTVFLRVRAKRGNLTSKW
VGLEPFQHYENVTVGPPKNISVTPGKGSLVIHFSPPFDVFHGATFQYLVHYWEKSETQQE
QVEGPFKSNSIVLGNLKPYRVYCLQTEAQLILKNKKIRPHGLLSNVSCHETTANASARLQ
QVILIPLGIFALLLGLTGACFTLFLKYQSRVKYWFQAPPNIGSSLFKGPRDYNPISSTICHLT
NESDGHTTSLYGIGFGPFIITNKHLFRRNNGTLLVQSLHGVFKVKNTTTLQQHLIDGRDMI
IIRMPKDFPPFPQKLKFREPQREERICLVTTNFQTKSMSSMVSDTSCTFPSSDGIFWKHWIQ
TKDGQCGSPLVSTRDGFIVGIHSASNFTNTNNYFTSVPKNFMELLTSQEAQQWVSGWRL
NADSVLWGGHKVFMSKPEEPFQPVKEATQLMNELVYSYPYDVPDYA
SEQ ID NO: 12 (IL10RB-linker-TEV (Mus musculus)):
MSWAPSVAGWLGGFLLVPALGMIPPPEKVRMNSVNFKNILQWEVPAFPKTNLTFTAQY
ESYRSFQDHCKRTASTQCDFSHLSKYGDYTVRVRAELADEHSEWVNVTFCPVEDTIIGPP
EMQIESLAESLHLRFSAPQIENEPETWTLKNIYDSWAYRVQYWKNGTNEKFQVVSPYDS
EVLRNLEPWTTYCIQVQGFLLDQNRTGEWSEPICERTGNDEITPSWIVAIILIVSVLVVFLF
LLGCFVVLWLIYKKTKHTFGSSLFKGPRDYNPISSTICHLTNESDGHTTSLYGIGFGPFIITN
KHLFRRNNGTLLVQSLHGVFKVKNTTTLQQHLIDGRDMIIIRMPKDFPPFPQKLKFREPQ
REERICLVTTNFQTKSMSSMVSDTSCTFPSSDGIFWKHWIQTKDGQCGSPLVSTRDGFIVG
IHSASNFTNTNNYFTSVPKNFMELLTSQEAQQWVSGWRLNADSVLWGGHKVFMSKPEE
PFQPVKEATQLMNELVYSYPYDVPDYA
SEQ ID NO: 13 (TNFαR1-linker-TEV (Homo sapiens)):
MGLSTVPDLLLPLVLLELLVGIYPSGVIGLVPHLGDREKRDSVCPQGKYIHPQNNSICCTK
CHKGTYLYNDCPGPGQDTDCRECESGSFTASENHLRHCLSCSKCRKEMGQVEISSCTVD
RDTVCGCRKNQYRHYWSENLFQCFNCSLCLNGTVHLSCQEKQNTVCTCHAGFFLRENE
CVSCSNCKKSLECTKLCLPQIENVKGTEDSGTTVLLPLVIFFGLCLLSLLFIGLMYRYQRW
KSKLYSIVCGGSSLFKGPRDYNPISSTICHLTNESDGHTTSLYGIGFGPFIITNKHLFRRNNG
TLLVQSLHGVFKVKNTTTLQQHLIDGRDMIIIRMPKDFPPFPQKLKFREPQREERICLVTT
NFQTKSMSSMVSDTSCTFPSSDGIFWKHWIQTKDGQCGSPLVSTRDGFIVGIHSASNFTNT
NNYFTSVPKNFMELLTSQEAQQWVSGWRLNADSVLWGGHKVFMSKPEEPFQPVKEAT
QLMNELVYSYPYDVPDYA
SEQ ID NO: 14 (TRE-H2B-GFP):
MPEPAKSAPAPKKGSKKAVTKAQKKGGKKRKRSRKESYSIYVYKVLKQVHPDTGISSK
AMGIMNSFVNDIFERIAGEASRLAHYNKRSTITSREIQTAVRLLLPGELAKHAVSEGTKAI
TKYTSAKDPPVATMVSKGEELFTGVVPILVELDGDVNGHKFSVSGEGEGDATYGKLTLK
FICTTGKLPVPWPTLVTTLTYGVQCFSRYPDHMKQHDFFKSAMPEGYVQERTIFFKDDG
NYKTRAEVKFEGDTLVNRIELKGIDFKEDGNILGHKLEYNYNSHNVYIMADKQKNGIKV
NFKIRHNIEDGSVQLADHYQQNTPIGDGPVLLPDNHYLSTQSALSKDPNEKRDHMVLLEF
VTAAGITLGMDELYK
SEQ ID NO: 15 (TRE-GFP):
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEG
DTLVNRIELKGIDFKEDGNILGHKLEYNYNSHNVYIMADKQKNGIKVNFKIRHNIEDGSV
QLADHYQQNTPIGDGPVLLPDNHYLSTQSALSKDPNEKRDHMVLLEFVTAAGITLGMDE
LYK

1. A reporter system for identifying a cytokine receptor modulator, the system comprising:
(a) a first fusion polypeptide comprising:
(i) a first domain comprising a first subunit of a cytokine receptor;
(ii) a second domain comprising at least a transcription activation domain and a DNA-binding domain of a transcription factor; and
(iii) a linker linking the first and second domain, wherein the linker comprises a protease cleavage site between the first and second domain; and
(b) a second fusion polypeptide comprising:
(i) a first domain comprising a second subunit of the cytokine receptor;
(ii) a second domain comprising at least a catalytic domain of a protease; and
(iii) a linker linking the first and second domain; and
(c) a reporter gene operably linked to a transcription factor regulatory element (TFRE),
wherein the DNA-binding domain of a transcription factor recognizes and binds the TFRE.

2. The reporter system of claim 1, wherein the linker linking the first and second domain of the first fusion polypeptide or second fusion polypeptide is a flexible linker.

3. (canceled)

4. The reporter system of claim 1, wherein the cytokine receptor is selected from the group consisting of: interleukin-17 receptor (IL-17R); IL-10R; interferon γ receptor (IFNγR); tumor necrosis factor receptor-1 (TNFR-1); and TNFR-2.

5. The reporter system of claim 4, wherein the first and second subunit of the cytokine receptor are selected from IL-17R A subunit (IL-17RA), IL-17R B subunit (IL-17RB) and IL-17R C subunit (IL-17RC).

6. The reporter system of claim 5, wherein the first subunit of the cytokine receptor is IL-17RA.

7. The receptor system of claim 6, wherein the second subunit of the cytokine receptor is IL-17RB or IL-17RC.

8. The reporter system of claim 5, wherein the first subunit of the cytokine receptor is IL-17RB or IL-17RC.

9. The receptor system of claim 8, wherein the second cytokine receptor domain IL-17RA.

10. The reporter system of claim 1, wherein the transcription factor is selected from the group consisting of: tetracycline-controlled transactivator (tTA), rtTA, LexA and VP16.

11. (canceled)

12. The reporter system of claim 1, wherein the protease is selected from the group consisting of: tobacco etch virus (TEV), thrombin, tissue plasminogen activator (tPA) serine proteases, trypsin, coagulation factor proteases, endopeptidase, neurotrypsin, chymotrypsin, mannan-binding lectin-associated serine proteases, cathepsin, furin, granzyme A, granzyme B, granzyme M, proteinase, and spinesin.

13. (canceled)

14. The reporter system of claim 1, wherein the first subunit of a cytokine receptor is IL-17RA and the transcription factor is tTA.

15. The T reporter system of claim 1, wherein the first subunit of a cytokine receptor is IL-17RB or IL17RC and the protease is TEV.

16. The reporter system of claim 1, wherein the reporter gene is operably linked to an inducible promotor.

17. The reporter system of claim 1, wherein the reporter gene is selected from the group consisting of: green fluorescent protein (GFP), cyan fluorescent protein (CFP), red fluorescent protein (RFP), β-galactosidase (lacZ), chloramphenicol acetyltransferase (cat), and luciferase (luc).

18. A cell comprising the reporter system of claim 1.

19. A polynucleotide encoding at least two of the first fusion polypeptide, the second fusion polypeptide and the reporter gene of claim 1.

20. (canceled)

21. The cell of claim 18, wherein the cell is selected from the group consisting of hepatocytes, fibroblasts, neurons, adipocytes, kidney cells and immune cells.

22. A method for identifying a test agent that modulates an immune response, the method comprising:
(i) contacting a cell with a test agent, wherein the cell comprises a reporter system of claim 1; and
(ii) detecting an expression level of the reporter gene, and
wherein an increase in the expression level of the reporter gene relative to a control or reference level indicates the agent modulates an immune response.

23. The method of claim 22, wherein said detecting the expression level of the reporter gene comprises spectroscopic, photochemical, biochemical, immunochemical, electrical, optical chemical detection, fluorescence detection, luminescence detection, chemilluminescence detection, immunofluorescence detection or FRET detection.

24. (canceled)

25. The method of claim 22, wherein the test agent is selected from the group consisting of small molecules, peptides, polypeptides, oligonucleotides, and polynucleotides, and lipids.
